(19) United States
(12) Patent Application Publication
(10) Pub. No.: US 2022/0160804 A1
(43) Pub. Date: May 26, 2022

(54) USE OF LAURUS NOBILIS EXTRACT FRACTIONS TO PROTECT AGAINST AIR POLLUTION RELATED DISEASES

(71) Applicant: DSM IP Assets B.V., Heerlen (NL)

(72) Inventors: Igor BENDIK, Kaiseraugst (CH); Bettina BOEHLENDORF, Kaiseraugst (CH); Pascale FUCHS BOSSERT, Kaiseraugst (CH); Hubert Paul HUG, Kaiseraugst (CH); Nathalie RICHARD, Kaiseraugst (CH); Guido WAHL, Kaiseraugst (CH)

(21) Appl. No.: 17/426,341

(22) PCT Filed: Jan. 23, 2020
(86) PCT No.: PCT/EP2020/051545

§ 371 Date: Jul. 28, 2021

(30) Foreign Application Priority Data
Jan. 30, 2019 (EP) 19154465.9

(51) Int. Cl. A61K 36/54; A61K 31/365; A61K 31/355; A61K 31/05; A61K 31/593; A61K 36/81; A61K 31/07; A61K 31/202; A61P 11/00

(52) U.S. Cl. CPC A61K 36/54; A61K 31/365; A61K 31/355; A61K 31/05; A61P 11/00; A61K 36/81; A61K 31/07; A61K 31/202; A61K 31/593

(57) ABSTRACT

The present invention relates to systemic detoxification and chronic inflammation by using extract fractions of Laurus nobilis, that lead to a decrease of anti-inflammatory markers in human lung cell lines and the activation of the Nrf2 pathway, which is a crucial element in intracellular detoxification pathways, decreases the expression of inflammatory cytokines. Therefore, the extract can be used to reduce the adverse effects of air pollution generally (and especially of particulate air pollution), which includes: cardiovascular problems, respiratory diseases, and chronic inflammation of tissues that come into contact with air borne particles.

### BRIEF DESCRIPTION OF THE INVENTION

The present invention relates to systemic detoxification by using fractions of a Laurus nobilis (bay leaf, bay laurel) extract.

### BACKGROUND OF THE INVENTION

Air pollution has been associated with morbidity and mortality mainly due to pulmonary and cardiovascular diseases.

Inflammation is a key process in the development of the diseases induced by the particulate matter of air pollution. Interleukin-8 (IL-8) is part of the innate immune system and important in the initiation of an immune response, but overstimulation and the resulting dysfunction of the recruited neutrophils within airways can result in the release of pro-inflammatory molecules resulting in the damage rather than protection of lung tissue.

Interleukin-6 (IL-6) is secreted by T-lymphocytes and macrophages and helps also to stimulate an immune response. IL-6 inhibits the actions of tumor necrosis factor α (TNF-α) and interleukin-1 (IL-1). It has been mainly connected with anti-inflammatory action but also some pro-inflammatory functions. Therefore, the benefit on its inhibition depends on the state of the infection. In chronic inflammation, it is helpful to decrease the expression of IL-6.

Monocyte chemoattractant protein-1 (MCP-1) recruits monocytes, memory T-lymphocytes and dendritic cells to the site of inflammation. Also, in chronic inflammation or inflammation mediated by air pollution it may be of advantage to decrease MCPO-1 expression.

Prostaglandin E2 (PGE2) is an inflammatory cytokine that increase pain caused by other inflammation mediators like bradykinin or histidine. It is also with other cytokines involved in the induction of fever. In addition is has other complex functions in many tissues. PGE2 is accepted as a general marker for inflammation.

An enhancement of the cellular detoxification pathway is considered to be helpful in conditions such as ageing, cardiovascular diseases, and lung diseases such as chronic obstructive pulmonary disease (COPD). Similarly, an enhancement of the cellular detoxification pathway should improve disorders caused by air pollution. Nuclear factor erythroid 2-related factor 2 (Nrf2) is a transcription factor that activates genes coding for detoxifying proteins. In its inactive state, it is part of a cytoplasmic complex with Kelch-like ECH-associated protein 1 (KEAP), a 69 kDa sensor protein that contains 27 cysteine residues and that acts as a dimer to bind both, Nrf2 and E3 ubiquitin ligase Cul3.

Nrf2 belongs to a cap ‘n’ collar family of basic leucine zipper transcription factors. Nrf2 becomes activated through modification of the SH-groups of KEAP1 and translocation into the nucleus where it binds, together with small MAF proteins to its anti-oxidative response element (ARE) in the promoters of its target genes. Target genes of Nrf2 are involved in anti-oxidative responses, phase II reactions and transport. In human COPD patients, it has been shown that an activation of Nrf2 can restore phagocytosis by alveolar macrophages.

Various Nrf2 activators are under development as pharmaceuticals. Bardoxolone methyl, a synthetic oleanane triterpenoid compound, is under clinical investigation for the treatment of pulmonary diseases. Also, the synthetic triterpenoid RTA 408 that possesses anti-oxidative and anti-inflammatory activities has been topically applied on human skin and is well tolerated by healthy human volunteers.

Aging is partly due to oxidative stress, i.e. oxidation and thereby damage of cellular molecules. Many chronic diseases are associated with aging. Since Nrf2 is a crucial factor for detoxification and for anti-oxidative host defenses it may help in slowing down aging. This has been shown in different well-established animal models and it is suspected that in long-lived humans Nrf2 is constitutively activated.

Broccoli sprouts rich in glucoraphanin, the precursor of the Nrf2-activator sulforaphane, attenuated nasal allergic responses to diesel exhaust particles. In a recent human intervention study, it was shown that broccoli sprouts enhanced the detoxification of some airborne pollutants, where a higher occurrence of glutathione-derived conjugates of benzene and acrolein, i. e. phase-II metabolites, could be shown in urine (Egner et al 2014. Cancer Prev Res Published OnlineFirst Jun. 9, 2014).

It is desirable to have natural compounds or extracts which can be used as a nutraceutical, pharmaceutical or food additive that could work via the Nf2 pathway to protect against air pollution.

### DETAILED DESCRIPTION OF THE INVENTION

It has been found, in accordance with this invention that fractions of a Laurus nobilis extract are potent Nrf2 pathway activators, and as such they can be used as general detoxification agents, similar to sulforaphane. We have found that these extract fractions have anti-inflammatory and Nrf2-activating activities in human lung cell lines treated with Diesel particulate matter. Preferably, the fractions can be used to protect the heart, lungs and respiratory system of a person or animal exposed to, or at risk of exposure to air pollution, and especially particulate air pollution.

In addition to the above, we have identified the compounds which are the active ingredients in the Laurus nobilis extracts, detailed below. Thus, these compounds or extracts enriched in these compounds can be used to protect the heart, lungs and respiratory system of a person or animal exposed to, or at risk of exposure to air pollution, and especially particulate air pollution.

We have shown that L. nobilis extract fractions can reduce Diesel particulate matter induced pro-inflammatory cytokines and can activate the transcription factor Nrf2. Nrf2 is a crucial element in intracellular detoxification pathways and decreases the expression of inflammatory cytokines. Therefore, the Laurus nobilis extract fractions can be used to reduce the adverse effects of air pollution generally (and especially of particulate air pollution), which includes: cardiovascular problems, respiratory diseases, and chronic inflammation of tissues that encounter air borne particles.

Anti-diabetic, anti-inflammatory, and anti-hyperlipidemic properties of the leaves of Laurus nobilis have been detected (Bower A et al 2016 Crit Rev Food Sci Nutr. 2016 56(16):2728-46). To date, no effects of Laurus nobilis in air pollution related diseases are described. For the first time, we found anti-inflammatory and Nrf2-activating activities in human lung cell lines treated with Diesel particulate matter.

The active ingredients have been identified as follows:

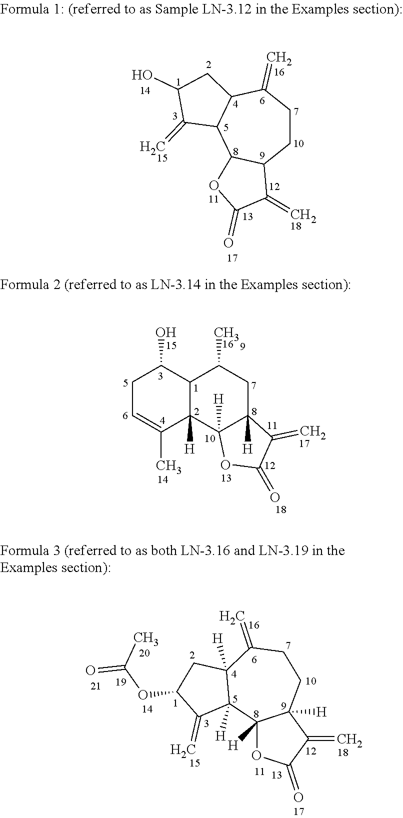

Thus another embodiment of this invention is a compound selected from the group consisting of: Formula I, Formula 2, Formula 3 and mixtures of at least two of these compounds, in the manufacture of a medicament for the use in protecting against harmful effects of air pollution.

Another embodiment of this invention is a Laurus nobilis extract enriched in at least one compound selected from the group consisting of Formula 1, 2, and 3 and mixtures of at least two of these compounds. Such enriched extracts can be administered to protect, ameliorate, or lessen the risk of cardiovascular and/or respiratory adverse condition resulting from the exposure to air pollution, preferably particulate matter air pollution, wherein the adverse condition is selected from the group consisting of: premature death in people who have heart or lung disease, non-fatal heart attacks, irregular heartbeat, asthma or aggravated asthma, decreased lung function, acute exacerbation of chronic obstructive pulmonary disease (COPD), and increased respiratory symptoms, such as irritation of the airways, coughing and/or difficulty breathing.

### Definitions

“Healthy person” means a person who a) has not been diagnosed with, or experiences symptoms of any of the following diseases or conditions: cardiovascular disease (including having had a non-fatal heart attack, irregular heartbeat, and impaired circulatory system), diabetes type 2, and respiratory disease, asthma or aggravated asthma, decreased lung function, or other conditions which result in difficulty breathing).

“Particulate air pollution” means which air which contains particles which are classified as nanoparticles, or have a particle size of PM_{2.5} or less. These size particles can be the result of “natural sources” such as volcanic emission, dust storms, forest fires, smoke from grassland fires and the like, or as a result of human activity such as automotive emissions, manufacturing emissions or other activities, including smoking.

“Cardiovascular health” is defined as the absence of conditions associated with abnormal cardiovascular functioning, such as: arthrosclerosis, myocardial infarction, stroke, thrombosis, peripheral artery disease, or decreased cerebral blood flow, and diabetes (Type I or Type 2) and its associated cardiovascular problems.

“Respiratory health” is defined as the absence of conditions associated with abnormal respiratory functioning, such as: asthma, emphysema, bronchitis, chronic obstructive pulmonary disease (COPD), hay-fever type allergies, coughs due to irritations, pulmonary infections, common cold symptoms and chronic sinusitis.

“Air pollution”, as used herein, refers to conditions where potentially harmful particulates, biological molecules or other substances have been introduced into the air. Examples of categories of pollutants include:
Sulfur oxides such as those produced as a result of coal and petroleum combustion;
Nitrogen oxides such as those produced from high temperature combustion, including nitrogen dioxide (one of the more prominent air pollutants, it is a reddish brown gas with a characteristic sharp odor);
Carbon monoxide which can be produced by incomplete combustion of fuel and vehicular exhaust;
Volatile organic compounds can include methane- or non-methane type compounds and are often referred to as greenhouse gases;
Particulates (also called particulate matter or PM) which are small solid or liquid particles which are suspended in the atmosphere. Origins may be “natura” such as from volcanic emissions, dust storms, or forest and grassland fires, or may be a result of human activities.
Pollution in the form of soot, gases and other matter which are in the form of tiny particles, termed “respirable particulate matter”. Respirable particulate matter is categorized by size, such as below 10 or 2.5 microns aerodynamic diameter (PM₁₀ or PM_{2.5}, respectively), or as nanoparticles (less than 100 nm diameter, or PM_{0.1}). These particles often come from vehicle emissions, particularly diesel fuel, or from diesel-powered machinery.
“Ameliorating the risk” of an adverse conditions means: protecting against the occurrence of the condition; preventing the occurrence of the condition; delaying the onset of a condition; lessening the severity of a condition that has already occurred; shortening the time that the condition persists; and/or elimination of the condition.

“Enriched extract” means an extract containing at least one compound of Formula 1, Formula 2 and/or Formula 3 in a weight % which is at least 10% higher than that found in a Laurus nobilis plant.

Particulates from human activities are linked to many health hazards, including heart disease and adverse respiratory conditions, including lung cancer.

Cigarette smoke also contains PMs as well as other chemicals which are also found in polluted air. Thus, another aspect of this invention is the use of a Laurus nobilis extract fraction, enriched extract, or at least one compound selected from the group of Formula 1, 2, and 3, or and mixtures of at least two of these compounds to protect a person exposed or at risk of exposure to cigarette smoke. Another aspect is a method of lessening the risk of adverse conditions in a person exposed to cigarette smoke comprising administering to the person at risk an effective amount of fractions of a Laurus nobilis extract, enriched extract, or at least one compound selected from the group of Formula 1, 2, and 3, or and mixtures of at least two of these compounds.

PM includes dust, dirt, soot and smoke. Particles termed “inhalable coarse particles” have diameters larger than 2.5 micrometers, but smaller than 10 micrometers. “Fine particles” are smaller, having diameters less than 2.5 micrometers. They are typically responsible for reduced visibility and haze. Many of the fine particles are “secondary particles”, which are the end products of chemical reactions in the atmosphere which occur when sulfur dioxides and nitrogen oxides are emitted by power plants, automobiles and other industrial activities. Fine particles are particularly troublesome as they can get deep into the lungs and the bloodstream and can potentially cause serious health problems, including:
Premature death in people who have heart or lung disease,
Non-fatal heart attacks
Irregular heartbeat
Asthma or aggravated asthma
Decreased lung function
Acute exacerbation of chronic obstructive pulmonary disease (COPD)
Increased respiratory symptoms, such as irritation of the airways, coughing and/or difficulty breathing.

Thus another aspect of this invention is the use of fractions of a Laurus nobilis extract enriched extract, or at least one compound selected from the group of Formula 1, 2, and 3, or and mixtures of at least two of these compounds to protect, ameliorate, or lessen the risk of cardiovascular and/or respiratory adverse condition resulting from the exposure to air pollution, preferably particulate matter air pollution, wherein the adverse condition is selected from the group consisting of: premature death in people who have heart or lung disease, non-fatal heart attacks, irregular heartbeat, asthma or aggravated asthma, decreased lung function, acute exacerbation of chronic obstructive pulmonary disease (COPD), and increased respiratory symptoms, such as irritation of the airways, coughing and/or difficulty breathing.

Another aspect is a method of lessening the risk of adverse conditions in a person exposed to air pollution, comprising administering fractions of a Laurus nobilis extract, enriched extract, or at least one compound selected from the group of Formula 1, 2, and 3 and mixtures of at least two of these compounds to a person in need thereof and wherein the adverse condition is selected from the group consisting of: premature death in people who have heart or lung disease, non-fatal heart attacks, irregular heartbeat, asthma or aggravated asthma, decreased lung function, acute exacerbation of chronic obstructive pulmonary disease (COPD), and increased respiratory symptoms, irritation of the airways, coughing and/or difficulty breathing.

Another aspect of this invention is a method of protecting, ameliorating or lessening the risk of cardiovascular and/or respiratory adverse condition resulting from the exposure to air pollution, preferably particulate matter air pollution, wherein the adverse condition is selected from the group consisting of: premature death in people who have heart or lung disease, non-fatal heart attacks, irregular heartbeat, asthma or aggravated asthma, decreased lung function, acute exacerbation of chronic obstructive pulmonary disease (COPD), and increased respiratory symptoms, such as irritation of the airways, coughing and/or difficulty breathing, comprising administering a fraction of a Laurus nobilis extract, an enriched extract or at least one compound selected from the group of Formula 1, 2, and 3, or and mixtures of at least two of these compounds.

Combinations with Other Active Ingredients

Fractions of a Laurus nobilis extract, enriched extract, or at least one compound selected from the group of Formula 1, 2, and 3, or and mixtures of at least two of these compounds of this invention may be combined with other active ingredients to make a composition which has beneficial results. Examples of further active ingredients include Vitamin E, water soluble tomato extract, resveratrol, Vitamin D, 25-hydroxy vitamin D3, hydroxytyrosol, polyunsaturated fatty acids (PUFAs), Vitamin A and mixtures thereof. Thus, this invention also includes the following combination of ingredients:
Fractions of a Laurus nobilis extract, enriched extract, or at least one compound selected from the group of Formula 1, 2, and 3, or mixtures of at least two of these compounds and Vitamin E;
Fractions of a Laurus nobilis extract, enriched extract, or at least one compound selected from the group of Formula 1, 2, and 3, or mixtures of at least two of these compounds and water-soluble tomato extract (such as FRUITFLOW® available from DSM Nutritional Products, Switzerland);
Fractions of a Laurus nobilis extract, enriched extract, or at least one compound selected from the group of Formula 1, 2, and 3, or mixtures of at least two of these compounds and resveratrol;
Fractions of a Laurus nobilis extract, enriched extract, or at least one compound selected from the group of Formula 1, 2, and 3, or mixtures of at least two of these compounds and Vitamin D;
Fractions of a Laurus nobilis extract, enriched extract, or at least one compound selected from the group of Formula 1, 2, and 3, or mixtures of at least two of these compounds and 25-OH Vitamin D3
Fractions of a Laurus nobilis extract, enriched extract, or at least one compound selected from the group of Formula 1, 2, and 3, or mixtures of at least two of these compounds and hydroxytyrosol;
Fractions of a Laurus nobilis extract, enriched extract, or at least one compound selected from the group of Formula 1, 2, and 3, or mixtures of at least two of these compounds and Polyunsaturated fatty acids (PUFAs); and
Fractions of a Laurus nobilis extract, enriched extract, or at least one compound selected from the group of Formula 1, 2, and 3, or mixtures of at least two of these compounds and Vitamin A.

In each of the above cases, the amount of the fractions of a Laurus nobilis extract, enriched extract, or at least one compound selected from the group of Formula 1, 2, and 3, or mixtures of at least two of these compounds is as detailed in this specification, and the amount of the second ingredient is present in an amount which is the maximum daily amount known in the art for each ingredient.

### Dosages

A recommended daily dose of a Laurus nobilis extract would be up to 2 grams/day for an adult. For enriched extracts, the amount would be enough to provide from 0.1 mg to 10 mg of the active ingredient(s) per day. For compounds of Formula 1, 2, or 3 as a sole active ingredient, a daily dose is from 0.1 mg to 10 mg; preferably from 0.5 to 8 mg per day and more preferably from 1-6 mg per day. For combinations of the active ingredients, the dosages may be adjusted so that the dosages of the combined ingredients are from at least 0.1 to 10 mg per day, but should not exceed 30 mg per day.

If desired, the daily intake can be divided into two or more dosages, such as twice a day tablets. For non-human animals, the human dosages above can be adjusted to the animal's body weight.

### Formulations

The composition of the present invention is preferably in the form of nutritional composition, such as fortified food, fortified feed, or fortified beverages, or in form of fortified liquid food/feed (such as drinks, or shots), pills or capsules for animals including humans.

The dietary and pharmaceutical compositions according to the present invention may be in any galenic form that is suitable for administering to the animal body including the human body, especially in any form that is conventional for oral administration, e.g. in solid form, such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be encapsulated in hard or soft-shell capsules, whereby the capsules feature e.g. a matrix of (fish, swine, poultry, cow) gelatin, plant proteins or lignin sulfonate. Examples for other application forms are forms for transdermal, parenteral or injectable administration. The dietary and pharmaceutical compositions may be in the form of controlled (delayed) release formulations. The compositions of the present invention are not administered topically, such as application to the nasal passage.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

Examples of food are cereal bars, dairy products, such as yoghurts, and bakery items, such as cakes and cookies. Examples of fortified food are cereal bars, and bakery items, such as bread, bread rolls, bagels, cakes and cookies. Examples of dietary supplements are tablets, pills, granules, dragées, capsules and effervescent formulations, in the form of non-alcoholic drinks, such as soft drinks, fruit juices, lemonades, near-water drinks, teas and milk-based drinks, in the form of liquid food, such as soups and dairy products (muesli drinks).

Beverages encompass non-alcoholic and alcoholic drinks as well as liquid preparations to be added to drinking water and liquid food. Non-alcoholic drinks are e.g. soft drinks, sport drinks, fruit juices, vegetable juices (e.g. tomato juice), lemonades, teas and milk-based drinks. Liquid foods are e.g. soups and dairy products (e.g. muesli drinks).

In addition to the fractions of a Laurus nobilis extract, enriched extract, or at least one compound selected from the group of Formula 1, 2, and 3, or mixtures of at least two of these compounds, pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum Arabic, vegetable oils, polyalkylene glycols, flavoring agents, preservatives, stabilizers, emulsifying agents, buffers, lubricants, colorants, wetting agents, fillers, and the like.

The following non-limiting Examples are presented to better illustrate the invention.

### Example 1

### Activation of Nrf2 Pathway

### Methods:

### Luciferase Reporter Assay Using H4IIE-ARE8L Cells:

H4IIE-ARE8L cells are a rat hepatoma cell line that is stably transfected with a luciferase reporter gene, which is controlled by eight times repeated anti-oxidative response elements (ARE) (Kratschmar D V, et al 2012. PloS One. 2012; 7 (5): e36774).

The medium for H4IIE-ARE8L cells was Dulbecco's Modified Eagle Medium (DMEM) high glucose containing heat inactivated 10% fetal bovine Serum (FBS). The media was exchanged every two to three-days. The DMEM assay medium used charcoal treated FBS (DMEMct).

The transactivation assay was performed in 96 well plates. The plates were seeded with approximately 40,000 cells per well in 100 μl DMEMct and incubated over night at 37° C. Then the test compounds were diluted in DMEDct and given to the cells as described below. The cells were incubated for at least further 16 h at 37° C. and 5% CO₂. Cells were equilibrated to room temperature. Lysis of the cells was done by adding 100 μL lysis solution, Steady-Glo® luciferase buffer according to the manufacturer (Promega) containing 0.5 mM DTT per well and incubated for 10 min at room temperature with gentle shaking. The luminescence was measured within 2 hours after incubation on a luminometer (Mithras, Berthold Technologies).

The positive control was 5 μM R-sulforaphane (LKT Laboratories Cat. 58046) in 0.5% DMSO, final concentrations respectively. The negative control were cells in 0.5% DMSO.

Cell survival assays of the H4IIE-ARE8L cells were performed with PrestoBlue® Cell Viability Reagent (ThermoFisher Scientific) according to the protocol of the manufacturer. Non-toxic concentrations of the extracts, fractions and single compounds were selected for the Nrf2-activity assay.

### Results:

We used a rat hepatoma cell line that was stably transfected with a construct that contains eight tandem ARE elements in front of a luciferase reporter gene (H4IIE-ARE8L) (Kratschmar D V, et al 2012. PloS One. 2012; 7(5):e36774). All extracts, fractions and pure compounds were tested for concentrations that induce toxicity. Non-toxic concentrations were then selected for treating cells as describes in the methods section.

In a primary screen of 1654 plant extracts, fractions, and pure compounds with our recombinant Nrf-2 activation assay four fractions of a Laurus nobilis extract showed a significant activation of Nrf-2 (Table 1). The whole Laurus nobilis extract was inactive, but some fractions of the extract showed activity. While not wishing to be bound by theory, we believe this can be explained as a) the active compound(s) are too diluted in the entire extract to register activity, but is more concentrated in the fraction; or b) the extract may contain an inhibitor that is removed during purification, although we believe this is less likely than a).

All extracts and fractions tested were not toxic to the H4IIE-ARE8L cells at the concentrations used.

TABLE 1
Nrf-2 activation of R-sulforaphane, DMSO, and fractions of a Laurus nobilis extract at
the indicated concentrations. Nrf-2 activity is given in relative units of luciferase fluorescence.
(For comparison, the value of the positive control R-sulforaphane is set to 1.0; n.s.: non-significant.)

| Compound | Conc. Final μg/ml | Average | Stdev |
|---|---|---|---|
| R-Sulforaphane | 4.55 | 13233 | 1299 |
| DMSO | 0.45 % | 2921 | 125 |
| C-0542-E-08 Laurus nobilis fraction | | 22235 | 12844 |
| C-0542-G-04 Laurus nobilis fraction | | 12376 | 2374 |
| C-0542-F-05 Laurus nobilis fraction | | 11229 | 2344 |
| C-0542-F-02 Laurus nobilis fraction | | 5923 | 1362 |

### Example 2

### Decrease of Inflammatory Markers Induced by Diesel Particles

### Methods:

The human bronchial epithelial cell line BEAS-2B was from ATCC (American Type Culture Collection, Manassas, Va.) and cultured in Bronchial Epithelial cell Growth Medium (BEGM, Lonza, Wakersville, Md.) in CellBIND® surface plastic flasks (Corning Inc., Corning, N.Y.). The adenocarcinomic human alveolar basal epithelial A549 cell line was obtained from ATCC and cultured in Kaighn's Modification of Ham's F-12 Medium (F-12K medium) (Life Technologies, USA), supplemented with 10% FBS (Sigma, Saint-Louis, Mo.). These cells were cultured at 37° C. in a humidified atmosphere containing 5% CO₂.

BEAS-2B cells were seeded in 12-well CellBIND® surface culture plates (Corning Inc.) at 3 to 4×10⁵ cells per well. A549 cells were seeded in 12-well plates at 2×10⁵ cells per well.

Diesel Particulate Matter (Standard Reference Material SRM 1650b, National Institute of Standards & Technology, NIST, Gaithersburg, Md.) at 80 mg/ml DMSO (100%) were sonicated for 5 min and thereafter diluted 400 fold in medium. This dilution was twofold further diluted for the assay.

After 24 h, cells were treated with the diluted Diesel Particulate Matter at 100 μg/ml and in the presence of different concentrations of Laurus Extracts, other plant extracts, fractions and compounds as indicated. The final DSMO concentrations were 0.175%. Untreated cells or cells treated with 0.175% DMSO were used as controls. After 24 h, cell supernatants were collected. The concentrations of IL-6 and IL-8 in the supernatants were determined by Luminex kits (BIO-RAD Laboratories, Hercules, Calif.) and used in the LiquiChip Workstation IS 200 (Qiagen, Hilden, Germany). The data were evaluated with the LiquiChip Analyser software (Qiagen).

Cell survival assays of the BEAS-2B and A549 cells were performed with AlamarBlue® Cell Viability Reagent (ThermoFisher Scientific) according to the protocol of the manufacturer. Non-toxic concentrations of the extracts, fractions and single compounds were selected for the assays. Secreted PGE2 was determined by Enzyme Immuno Assay (EIA) (Cayman Chemicals, Ann Harbor, Wis.).

Mean values, standard deviation and p-values with Student's t-test were calculated with Excel. P-values greater than 0.05 were considered as indication for significance.

### Results:

Fractions of a Laurus nobilis extract were tested for their ability to inhibit Diesel Particulate Matter (PM)-induced IL-6 secretion in human lung cell lines. Untreated BEAS-2B cells did not secrete IL-6 (Table 2, row 2). The solvent DMSO showed a slight decrease in IL-6 secretion of BEAS-2B cells and therefore, all values of the tested Laurus nobilis fractions have to be compared with the PM control in the presence of DMSO (Table 2, rows 1 and 3). TiO₂ particles did not lead to an increase in IL-6 secretion which shows that a physical effect of the particles is not responsible for the effects (Table 2, row 5). Lipopolysaccharide (LPS) a known inducer of IL-6 had a strong effect; it was over 40 times stronger than PM and DMSO (Table 2, rows 1 and 4).

TABLE 2
IL-6 secretion (pg/ml) of BEAS-2B cells treated with Diesel Particulate Matter (PM) in
the presence of compounds as indicated. PM concentration was always 100 μg/ml. The IL-6
concentration of the positive control PM with DMSO was set to 100% for comparison.

| | | IL-6 | Standard | % of PM | p-value |
|---|---|---|---|---|---|
| Treatment | Concentration | [pg/mL] | deviation | + DMSO | (t-test) |
| PM + DMSO | 0.175% | 84 | 4 | 100 | 1 |
| Untreated cells | | 1.6 | 0.1 | 1 | 0.001 |
| PM | | 119 | 4 | 141 | 0.012 |
| LPS | 10 μg/mL | 3760 | 424.3 | 4482 | 0.007 |
| TiO₂ | 100 μg/mL | 1.5 | 0.2 | 1 | 0.001 |

Fraction of extracts of Laurus nobilis were treated as shown in Table 3, below. The positive control, an extract of Withania somnifera WS-7.1, showed a significant decrease of IL-6 secretion on BEAS-2B cells (Table 3, row 5). None of the Laurus nobilis fractions showed a significant decrease.

TABLE 3
IL-6 secretion (pg/ml) of BEAS-2B cells treated with Diesel Particulate Matter (PM) in the
presence of different Laurus nobilis extract fractions. PM concentration was always 100 μg/ml.
The IL-6 concentration of the positive control PM with DMSO was set to 100% for comparison.

| | | IL-6 | Standard | % of PM | p-value |
|---|---|---|---|---|---|
| Treatment | Concentration | [pg/mL] | deviation | + DMSO | (t-test) |
| PM + DMSO | 0.175% | 61 | 5.1 | 100 | 1 |
| Untreated cells | | 0.9 | 0 | 1 | |
| PM | | 81.2 | 10.2 | 133 | 0.037 |
| LPS | 10 μg/mL | 6477 | 336 | 10612 | 0.000005 |
| PM + Withania somnifera | 25 μg/mL | 23.4 | 1.1 | 38 | 0.0002 |
| WS-7.1 | | | | | |
| PM + C-0542-C-02 | 1 μg/mL | 75.9 | 5.5 | 124 | 0.026 |
| PM + C-0542-D-01 | 1 μg/mL | 76.9 | 2.8 | 126 | 0.009 |
| PM + C-0542-D-05 | 1 μg/mL | 69.2 | 10.4 | 113 | 0.290 |
| PM + C-0542-E-02 | 1 μg/mL | 68 | 9.6 | 111 | 0.329 |
| PM + C-0542-E-03 | 1 μg/mL | 68.2 | 3.4 | 112 | 0.109 |
| PM + C-0542-E-04 | 1 μg/mL | 57.4 | 4.2 | 94 | 0.387 |
| PM + C-0542-E-07 | 1 μg/mL | 79.1 | 4.6 | 130 | 0.010 |
| PM + C-0542-E-08 | 1 μg/mL | 63.5 | 9.8 | 104 | 0718 |
| PM + C-0542-F-02 | 1 μg/mL | 75.4 | 9.9 | 123 | 0.089 |
| PM + C-0542-F-03 | 1 μg/mL | 77.3 | 5.7 | 127 | 0.021 |
| PM + C-0542-F-05 | 1 μg/mL | 91.3 | 9.1 | 150 | 0.007 |
| PM + C-0542-G-08 | 1 μg/mL | 85.9 | 8.7 | 141 | 0.013 |
| PM + C-0542-G-10 | 1 μg/mL | 94.7 | 4.2 | 155 | 0.001 |
| PM + NIG-014682 | 1 μg/mL | 67.3 | 7.1 | 110 | 0.282 |
| PM + NIG-008368 | 1 μg/mL | 67.7 | 15.4 | 111 | 0.516 |
| PM + NIG-006259 | 1 μg/mL | 71 | 7.5 | 116 | 0.130 |
| PM + V-00075-W-02 | 5 μg/mL | 90.8 | 7.2 | 149 | 0.004 |

IL-6 secretion in the presence of PM of the human lung cell line A549 was decreased by several Laurus nobilis fractions. The results are shown in Table 4, below. Significantly positive were fractions C-0542-E-04 (Row 11) and fraction C-0542-F-02 (row 14).

TABLE 4
IL-6 secretion (pg/ml) of A549 cells treated with Diesel Particulate Matter (PM) in the
presence of pure compounds and Laurus nobilis extract fractions as indicated. PM
concentration was always 100 μg/ml. The IL-6 concentration of the positive control PM
with DMSO was set to 100% for comparison.

| | | IL-6 | Standard | % of PM | p-value |
|---|---|---|---|---|---|
| Treatment | Concentration | [pg/mL] | deviation | + DMSO | (t-test) |
| PM + DMSO | 0.175% | 43.2 | 2.8 | 100 | 1 |
| Untreated cells | | 8 | 0.6 | 19 | 0.0005 |
| PM | | 51.2 | 0.4 | 118 | 0.033 |
| LPS | 10 μg/mL | 19.6 | 2.1 | 45 | 0.002 |
| PM + Withania somnifera | 25 μg/mL | 32.5 | 0.6 | 75 | 0.003 |
| WS-7.1 | | | | | |
| PM + C-0542-C-02 | 1 μg/mL | 48.3 | 0.3 | 112 | 0.035 |
| PM + C-0542-D-01 | 1 μg/mL | 46.3 | 2 | 107 | 0.191 |
| PM + C-0542-D-05 | 1 μg/mL | 47.3 | 5.8 | 109 | 0.333 |
| PM + C-0542-E-02 | 1 μg/mL | 45.2 | 3.2 | 105 | 0.464 |
| PM + C-0542-E-03 | 1 μg/mL | 48.3 | 5.8 | 112 | 0.240 |
| PM + C-0542-E-04 | 1 μg/mL | 37.5 | 0.3 | 87 | 0.026 |
| PM + C-0542-E-07 | 1 μg/mL | 40.5 | 1.9 | 94 | 0.241 |
| PM + C-0542-E-08 | 1 μg/mL | 42.8 | 1.8 | 99 | 0.848 |
| PM + C-0542-F-02 | 1 μg/mL | 28.3 | 2.3 | 65 | 0.002 |
| PM + C-0542-F-03 | 1 μg/mL | 45 | 2.9 | 104 | 0.472 |
| PM + C-0542-F-05 | 1 μg/mL | 43.7 | 2.7 | 101 | 0.813 |
| PM + C-0542-G-08 | 1 μg/mL | 40.5 | 2.7 | 94 | 0.301 |
| PM + C-0542-G-10 | 1 μg/mL | 40.3 | 1.5 | 93 | 0.193 |
| PM + NIG-014682 | 1 μg/mL | 49.3 | 1.5 | 114 | 0.030 |
| PM + NIG-008368 | 1 μg/mL | 48.5 | 1.5 | 112 | 0.044 |
| PM + NIG-006259 | 1 μg/mL | 45.4 | 5.3 | 105 | 0.556 |
| PM + V-00075-W-02 | 5 μg/mL | 42.8 | 1.2 | 99 | 0.847 |

Also, IL-8 secretion in the presence of PM of the human lung cell line A549 was decreased by several Laurus nobilis fractions. The results are shown in Table 5, below. Significantly positive were again fractions C-0542-E-04 (Row 11) and fraction C-0542-F-02 (Row 14).

TABLE 5
IL-8 secretion (pg/ml) of A549 cells treated with Diesel Particulate Matter (PM) in the
presence of Laurus nobilis extract fractions as indicated. PM concentration was always 100 μg/ml.
The IL-8 concentration of the positive control PM with DMSO was set to 100% for comparison.

| | | IL-8 | Standard | % of PM | p-value |
|---|---|---|---|---|---|
| Treatment | Concentration | [pg/mL] | deviation | + DMSO | (t-test) |
| PM + DMSO | 0.175% | 290 | 30.8 | 100 | 1 |
| Untreated cells | | 117 | 14.1 | 40 | 0.006 |
| PM | | 333 | 21.2 | 115 | 0.191 |
| LPS | 10 μg/mL | 406 | 50.9 | 140 | 0.046 |
| PM + Withania somnifera | 25 μg/mL | 192.7 | 24 | 66 | 0.012 |
| WS-7.1 | | | | | |
| PM + C-0542-C-02 | 1 μg/mL | 382 | 65.6 | 132 | 0.093 |
| PM + C-0542-D-01 | 1 μg/mL | 328 | 58.8 | 113 | 0.378 |
| PM + C-0542-D-05 | 1 μg/mL | 280.3 | 7.4 | 97 | 0.625 |
| PM + C-0542-E-02 | 1 μg/mL | 296.7 | 34.1 | 102 | 0.814 |
| PM + C-0542-E-03 | 1 μg/mL | 326.7 | 65.1 | 113 | 0.427 |
| PM + C-0542-E-04 | 1 μg/mL | 240.7 | 28.6 | 83 | 0.112 |
| PM + C-0542-E-07 | 1 μg/mL | 273.7 | 12.7 | 94 | 0.444 |
| PM + C-0542-E-08 | 1 μg/mL | 268 | 23.4 | 92 | 0.381 |
| PM + C-0542-F-02 | 1 μg/mL | 235.3 | 26.3 | 81 | 0.080 |
| PM + C-0542-F-03 | 1 μg/mL | 290 | 32.2 | 100 | 1 |
| PM + C-0542-F-05 | 1 μg/mL | 357.7 | 18.1 | 123 | 0.031 |
| PM + C-0542-G-08 | 1 μg/mL | 266.7 | 28.3 | 92 | 0.389 |
| PM + C-0542-G-10 | 1 μg/mL | 295.3 | 26.1 | 102 | 0.830 |
| PM + NIG-014682 | 1 μg/mL | 320.7 | 30 | 111 | 0.284 |
| PM + NIG-008368 | 1 μg/mL | 288.3 | 31.7 | 99 | 0.951 |
| PM + NIG-006259 | 1 μg/mL | 352.7 | 11.5 | 122 | 0.030 |
| PM + V-00075-W-02 | 5 μg/mL | 286 | 36.1 | 99 | 0.891 |

Then, MCP-1 secretion in the presence of PM of the human lung cell line A549 was measured with the same Laurus nobilis fractions. No significant changes could be observed.

### Example 3

### Identification of Active Ingredients

Four compounds were isolated from positive samples as discussed at the end of this Example and analyzed by NMR spectroscopy in order to elucidate structure. Results are shown below:

| Sample ID | Amount | Structure Proposal |
|---|---|---|
| LN-3.12 | 25 mg | 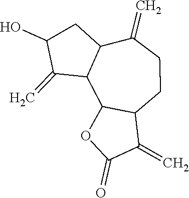 [SMILES: C=C1C(=O)OC2C1CCC(=C)C1CC(O)C(=C)C12] |
| LN-3.14 | 37 mg | 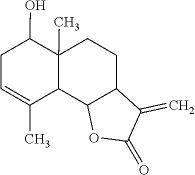 [SMILES: C=C1C(=O)OC2C1CCC1(C)C(O)CC=C(C)C21] |
| LN-3.16 | 23 mg | 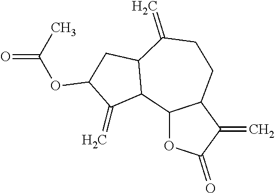 [SMILES: C=C1C(=O)OC2C1CCC(=C)C1CC(OC(C)=O)C(=C)C12] |
| LN-3.19 | 4 mg | 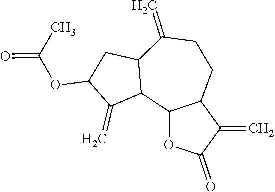 [SMILES: C=C1C(=O)OC2C1CCC(=C)C1CC(OC(C)=O)C(=C)C12] |

Spectra are given below:

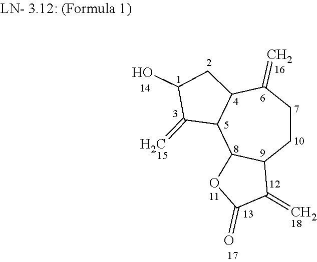 [SMILES: C=C1C(=O)OC2C1CCC(=C)C1CC(O)C(=C)C12]

The numbering system is used for assignment in Table 6, below.

TABLE 6
Characteristic ¹H and ¹³C assignment of the structure of Formula 1.

| Atom | δ (¹H)/ | Int. | Mult. | δ (¹³C)/ | | |
|---|---|---|---|---|---|---|
| # | ppm | (1H) | (¹H) | ppm | HMBC | NOESY |
| 1 | 4.53 | CH | tt | 74.3 | C2, C15, C3 | 2.26 |
| 2 | 2.26 | CH2 | m | 40.1 | C1, C6, C4 | 1.73, 2.97 |
| | 1.73 | | | | | 2.26, 2.52, 4.99 |
| 3 | — | C | — | 154.9 | — | — |
| 4 | 2.97 | CH | m | 44.9 | Ov. | Ov. |
| 5 | 2.88 | CH | tt | 50.6 | C4, C8, C15, | — |
| | | | | | C6, C3 | |
| 6 | — | C | — | 150.5 | — | — |
| 7 | 2.52 | CH2 | m | 35.9 | Ov. | Ov. |
| | 2.21 | | | | | 2.52, 2.32, 1.46 |
| 8 | 4.14 | CH | dd | 86.3 | C10, C3 | 1.46, 5.33 |
| 9 | 2.96 | CH | m | 46.6 | Ov. | Ov. |
| 10 | 2.32 | CH2 | m | 32.3 | C7, C9, C8, | 1.46, 2.52 |
| | 1.46 | | | | C12, C6 | 2.32, 2.52 |
| 12 | — | C | — | 142.1 | — | — |
| 13 | — | C | — | 172.6 | — | — |
| 15 | 5.28 | CH2 | t | 110.3 | C5, C1, C3 | 5.33 |
| | 5.33 | | | | | 5.28 |
| 16 | 4.94 | CH2 | m | 114.7 | C10, C7, C4, | 1.46, 2.52 |
| | 4.99 | | | | C5, C6 | 1.73 |
| 18 | 6.15 | CH2 | dd | 121.0 | C9, C12, C13 | 5.62 |
| | 5.62 | | | | | 6.15, 2.32 |

The stereochemistry of this structure could not be determined because of the overlap of the signals from the protons in C4, C5 and C9.

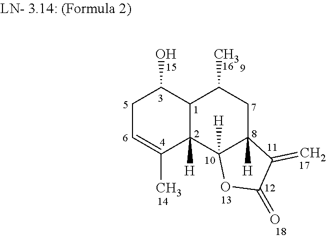

Formula 1. The numbering system is used for assignment in Table 7, below.

TABLE 7
Characteristic ¹H and ¹³C assignment of the structure in Formula 2:

| Atom | δ (¹H)/ | Int. | Mult. | δ (¹³C)/ | | |
|---|---|---|---|---|---|---|
| # | ppm | (¹H) | (¹H) | ppm | HMBC | NOESY |
| 1 | — | C | — | 40.8 | — | — |
| 2 | 2.40 | CH | Ov. | 50.9 | C16, C1, C4, | 3.63, 2.58 |
| | | | | | C10 | |
| 3 | 3.64 | CH | dd | 74.5 | C5, C16, C1, | 2.37, 1.35 |
| | | | | | C2 | |
| 4 | — | C | — | 132.9 | — | — |
| 5 | 2.01 | CH2 | m | 32.1 | C6, C3, C4, | 0.89, 2.37 |
| | 2.37 | | | | C1, C2 | 2.01 |
| 6 | 5.38 | CH | m | 121.4 | C14, C2, C3, | 2.37, 2.01, 1.85 |
| | | | | | C5 | |
| 7 | 2.12 | CH2 | m | 20.8 | C9, C8, C10 | 1.68 |
| | 1.68 | | | | | 2.12, 1.35, 0.89 |
| 8 | 2.58 | CH | m | 50.8 | C7, C2, C10, | 1.35, 2.40 |
| | | | | | C11, C17 | |
| 9 | 2.05 | CH2 | m | 34.2 | C16, C3, C1, | 1.35 |
| | 1.35 | | | | C7, C8 | 2.37, 2.58, 3.64, |
| | | | | | | 1.68 |
| 10 | 4.03 | CH | dd | 82.0 | C1, C2, C8, | 0.89, 1.68, 1.85 |
| | | | | | C7, C11 | |
| 11 | — | C | — | 139.0 | — | — |
| 12 | — | C | — | 171.0 | — | — |
| 14 | 1.85 | CH3 | m | 22.3 | C2, C4, C6 | 2.40, 4.03, 5.38 |
| 16 | 0.89 | CH3 | s | 10.1 | C9, C1, C2, | 4.03, 2.05, 1.67 |
| | | | | | C3 | |
| 17 | 6.01 | CH2 | d | 115.6 | C8, C11, C12 | 5.51 |
| | 5.51 | | | | | 6.01, 2.12 |

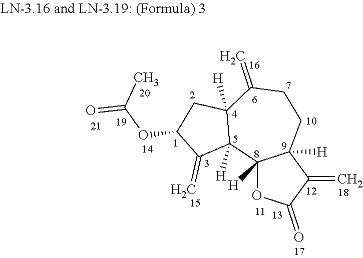 [SMILES: C=C1[C@@H]2[C@H]3OC(=O)C(=C)[C@@H]3CCC(=C)[C@@H]2C[C@H]1OC(C)=O]

Formula 3. The numbering system is used for assignment in Table 8, below.

TABLE 8
Characteristic ¹H and ¹³C assignment of the structure in Formula 3.

| Atom | δ (¹H)/ | Int. | Mult. | δ (¹³C)/ | | |
|---|---|---|---|---|---|---|
| # | ppm | (¹H) | (¹H) | ppm | HMBC | NOESY |
| 1 | 5.60 | CH | m | 74.8 | C2, C3, C15 | — |
| 2 | 2.47 | CH2 | m | 36.0 | C4, C5, C3, | 3.05, 1.87 |
| | 1.87 | | | | C6, C1 | 2.47 |
| 3 | — | C | — | 149.2 | — | — |
| 4 | 3.05 | CH | m | 44.3 | Ov. | 2.47, 2.22, 1.87 |
| 5 | 2.95 | CH | m | 49.9 | Ov. | 3.05 |
| 6 | — | C | — | 148.7 | — | — |
| 7 | 2.54 | CH2 | m | 34.7 | Ov. | 2.22, 2.35, 1.47 |
| | 2.22 | | | | | 2.54, 1.47 |
| 8 | 4.11 | CH | dd | 84.5 | C3, C5, C4, | 5.40, 1.47, 1.87, |
| | | | | | C9, C10 | 2.54 |
| 9 | 2.99 | CH | m | 44.9 | Ov. | 2.35 |
| 10 | 2.35 | CH2 | m | 30.7 | C6, C7, C9, | 1.47, 2.54 |
| | 1.47 | | | | C8, C12 | 2.22, 2.35, 2.54 |
| 12 | — | C | — | 140.1 | — | — |
| 13 | — | C | — | 170.7 | — | — |
| 15 | 5.40 | CH2 | t | 111.5 | C3, C5, C1 | 5.26 |
| | 5.26 | | | | | 5.40 |
| 16 | 4.97 | CH2 | br. s | 112.9 | C5, C4, C7, | 1.87, 2.54 |
| | | | | | C10, C6 | |
| 18 | 6.17 | CH2 | d | 119.4 | C9, C12, C13 | 5.63 |
| | 5.63 | | | | | 6.17, 2.35 |
| 19 | — | C | — | 171.1 | — | — |
| 20 | 2.11 | CH3 | s | 19.8 | C19, C1, C2 | — |

Ov. stands for overlapped.

Four isolated fractions of a Laurus nobilis extract are analyzed by NMR spectroscopy in order to elucidate their structure. The proposed structures were confirmed and characterized. LN-3.16 and LN-3.19 consist of the same compound. The proposed stereochemistry is based on NOESY experiments.

### EXPERIMENTAL

NMR experiments were performed on a Bruker Avance III NMR spectrometer operating at 600 MHz proton corresponding to 150 MHz carbon Larmor frequency and equipped with a cryogenically cooled 5 mm TCl probe. All experiments were carried out at 298 K.

Spectra were recorded in deuterated methanol for lock purposes. The chemical shifts were referenced to the solvent. Data were acquired using TopSpin 3.6 and processed with ACD/Labs running on a personal computer.

1. A composition comprising an active ingredient selected from the group consisting of:
a) Fractions of a Laurus nobilis extract,
b) a Laurus nobilis enriched extract, and
c) at least one compound selected from the group of Formula 1, 2, and 3, and mixtures of at least two of these compounds;
wherein Formula 1 is: 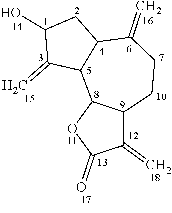 [SMILES: C=C1C(=O)OC2C1CCC(=C)C1CC(O)C(=C)C12]
Formula 2 is: 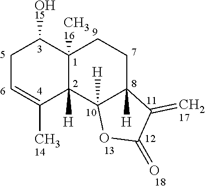
and Formula 3 is: 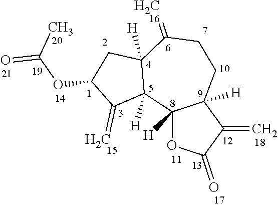
for use to prevent or ameliorate the adverse effects of air pollution.

2. A composition according to claim 1 wherein the air pollution is particulate air pollution.

3. A composition according to claim 1 wherein the adverse effect is selected from the group consisting of: cardiovascular problems, respiratory diseases, and chronic inflammation of tissues that come into contact with air borne particles.

4. A composition according to claim 1 wherein the particulate air pollution is from cigarette smoke.

5. A composition according to any claim 1 further comprising and active ingredient selected from the group consisting of: Vitamin E, water soluble tomato extract, resveratrol, Vitamin D, 25-hydroxy vitamin D3, hydroxytyrosol, polyunsaturated fatty acids (PUFAs), Vitamin A and mixtures thereof.

6. A nutraceutical, functional food, or food supplement comprising a composition according to claim 1.

7. A method of ameliorating the adverse effects of exposure to air pollution comprising administering an effective amount of a composition comprising an active ingredient selected from the group consisting of:
a) fractions of a Laurus nobilis extract,
b) a Laurus nobilis enriched extract, and
c) at least one compound selected from the group of Formula 1, 2, and 3, and mixtures of at least two of these compounds
wherein Formula 1 is: 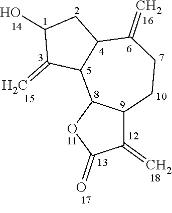 [SMILES: C=C1C(=O)OC2C1CCC(=C)C1CC(O)C(=C)C12]
Formula 2 is: 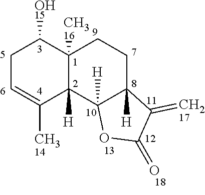
and Formula 3 is: 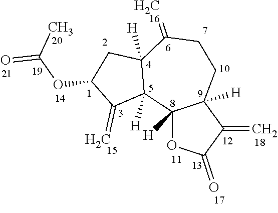
to a person or animal exposed to or at risk of exposure to air pollution.

8. A method according to claim 7 wherein the air pollution is particulate air pollution.

9. A method according to claim 7 wherein the adverse effect is selected from the group consisting of: cardiovascular problems, respiratory diseases, and chronic inflammation of tissues that come into contact with air borne particles.

10. A method according to claim 9 wherein the particulate air pollution is from cigarette smoke.

11. A method according to claim 7 further comprising and active ingredient selected from the group consisting of: Vitamin E, water soluble tomato extract, resveratrol, Vitamin D, 25-hydroxy vitamin D3, hydroxytyrosol, polyunsaturated fatty acids (PUFAs), Vitamin A and mixtures thereof.

12. A method according to claim 7 wherein the composition is a nutraceutical, functional food, or food supplement.
